(19) United States
(12) Patent Application Publication
(10) Pub. No.: US 2014/0128742 A1
(43) Pub. Date: May 8, 2014

(54) CANCER TISSUE DETECTION AND DIAGNOSIS USING LASER FLUORESCENCE

(71) Applicant: 2M Engineering Limited, (US)

(72) Inventor: Marcel F. Schemmann, Maria Hoop (NL)

(73) Assignee: 2M Engineering Limited, Veldhoven (NL)

(21) Appl. No.: 13/776,610

(22) Filed: Feb. 25, 2013

Related U.S. Application Data
(60) Provisional application No. 61/602,465, filed on Feb. 23, 2012.
(60) Provisional application No. 61/736,769, filed on Dec. 13, 2012.

(51) Int. Cl. A61B 5/00

(52) U.S. Cl. CPC A61B 5/0071; A61B 5/0075
USPC 600/476

(57) ABSTRACT

A laser diagnostic system includes multiple lasers providing incident light on a sample, the light modulated and cooperatively phased to stimulate red emissions from the sample; and a detector coupled to a spectrum analyzer, to detect both the incident light and the red emissions from the sample, and to provide a signal indicating detection of a pathology in the sample.

### PRIORITY

This application claims priority under 35 USC 119 to application serial no. U.S. 61/602,465 filed on Feb. 23, 2012, and under 35 USC 119 to application serial no. U.S. 61/736,769 filed on Dec. 13, 2012, each of which is incorporated herein by reference in its entirety.

### BACKGROUND

Cancer tissue may exhibit fluorescence when exposed to certain substances and subsequently to laser light. The fluorescence of the cancer tissue may be distinguishable from fluorescence of surrounding healthy tissue. However, conventional fluorescent diagnosis techniques for cancer tissue are prone to false positives or negatives due to being insufficiently sensitive and/or infused with noise signals.

### BRIEF DESCRIPTION OF THE DRAWINGS

In the drawings, the same reference numbers and acronyms identify elements or acts with the same or similar functionality for ease of understanding and convenience. To easily identify the discussion of any particular element or act, the most significant digit or digits in a reference number refer to the figure number in which that element is first introduced.

FIG. 1 illustrates fluorescence emission spectra of human plasma.

FIG. 2 illustrates intracellular distribution of porphyrins synthesized from the porphyrin prodrug 5-aminolaevulinic acid as determined by confocal microscopy.

FIG. 3 illustrates endogenous fluorescence from nonmelanoma skin cancer (T) and sounding normal skin (in) from two individuals at two different body sites.

FIG. 4 illustrates an embodiment of a diagnostic system to carry out procedures described herein.

### DETAILED DESCRIPTION

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other.

“Logic” refers to machine memory circuits, machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic.

Those skilled in the art will appreciate that logic may be distributed throughout one or more devices, and/or may be comprised of combinations memory, media, processing circuits and controllers, other circuits, and so on. Therefore, in the interest of clarity and correctness logic may not always be distinctly illustrated in drawings of devices and systems, although it is inherently present therein.

The techniques and procedures described herein may be implemented via logic distributed in one or more computing devices. The particular distribution and choice of logic is a design decision that will vary according to implementation.

In the following description “near 400 nm” means between 355 and 470 nm. “Near 500 nm” means between 470 and 610 nm. “Near 600 nm” means between 610 and 745 nm. The term “approximately” in conjunction with a particular peak emission wavelength refers to a range of wavelengths on both sides of the peak emission wavelength. The extent of this range will vary according to particular diagnostic situations, according to characteristics of the patient being evaluated, the nature of the cancer cells being screened for, and possibly other factors as well. Examples of such ranges include +−1 nm and +−3 nm, depending on conditions.

### Overview

Cancer tissue cells tend to create additional blood vessels to support their growth. The protein protoporphyrin (PpIX) is generally present and exhibits red photo fluorescence in the 600 nm range when exited with blue light in the 400 nm range. The absorption wavelength depends on the local conditions and by tuning an excitation laser wavelength an absorption wavelength is detected when the red emission is maximized. The red emission intensity can be an indication of affected tissue.

The PpIX sensitivity to excitation is wavelength dependent and the required wavelength and intensity are dependent on other chemicals present. This wavelength and intensity dependence on the environment may be exploited to detect the presence of cancer cells that are forming blood vessels. The cancer cells are detected by observing the red photo fluorescence at a matching excitation wavelength. The intensity of the red photo fluorescence will be higher near cancer cells. The matching excitation wavelength can depend on the person and type of illness and thus may not be known a priori. Preferably, an excitation laser system is used that can generate a range of wavelengths, for instance between 370 and 420 nm, where the protoporphyrin exhibits a sensitivity maximum.

The excitation may laser system may be applied in vivo, for instance via an endoscopic device, where the excitation wavelength is delivered via fiber and fiber-optic observation are used to visually or electronically observe the red photo fluorescence. The excitation laser wavelength may be adjusted over a range of at least 5 nm, in steps of at least 0.5 nm. The excitation laser wavelength may be tuned (adjusted) while simultaneously observing photo fluorescence at a red wavelength in the 630-690 nm range. The excitation laser wavelength may be tuned to obtain maximum emission at the photo fluorescence wavelength, and/or to obtain a good contrast between cancerous tissue and the surrounding tissue.

### Description of Example Designs

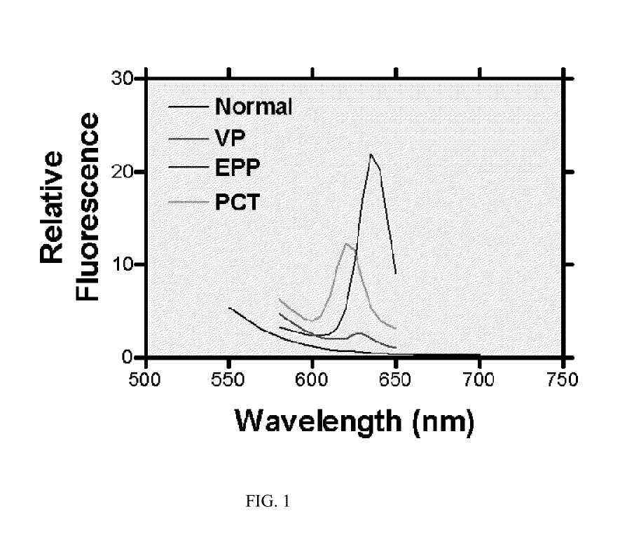

Erythropoietic protoporphyria and variegate porphyria may be distinguished from each other and from other porphyrias based on the fluorescence emission maxima of endogenous porphyrins. See for example FIG. 1 and Table 1.

TABLE 1
Fluorescence emission maxima used in the
preliminary screening diagnosis of porphyria.

| Porphyria | Emission maximum (nm) |
|---|---|
| Erythropoietic protoporphyria | 635 |
| Variegate porphyria | 626 |
| Porphyria cutanea tarda | 621 |
| Hereditary coproporphyria | |

To obtain the data samples graphed in FIG. 1, whole blood samples are centrifuged and plasma removed and diluted 1:10 in saline. A laser is applied having an emission wavelength of 405 nm. The signal from the non-porphyric subject (black line, FIG. 1) slopes from 550-650 nm due to fluorescence from non-porphyrin fluorophores. The other three samples are from subjects with porphyria and the red emission is evident.

The emission maximum of porphyria cutanea tarda and hereditary coproporphyria is heavily influenced by the predominance of uroporphyrin and coproporphyrin, whereas the emission maximum of erythropoietic protoporphyria is due to protoporphyrin IX (PpIX). Variegate porphyria also results in an overproduction of PpIX, however in this instance the porphyrin is bound to protein which shortens its emission to 626 nm. Aggregation and binding to metal can also alter the spectral properties of porphyrins. The effect of environment on the fluorescence emission maximum of PpIX can be seen in Tables 2a-2e.

TABLE 2a
Fluorescence data for PpIX (5 μM) in DMSO
Solvent: DMSO

| λex | Emission maxima (nm) | | | | | |
|---|---|---|---|---|---|---|
| (nm) | Peak 1 | Peak 2 | Peak 3 | Peak 4 | Peak 5 | ^{b}AUC |
| 368 | 634.5 | 663.5 | 684.5 | 701.0 | ND | 210.0 |
| 403 | 634.5 | 661.5 | 684.5 | 699.5 | ND | 372.0 |
| 408 | 634.5 | 663.5 | 686.0 | 691.0 | 702.5 | 370.6 |
| 416 | 634.5 | 662.5 | 686.5 | 702.0 | ND | 309.2 |
| 417 | 634.5 | 664.5 | 686.5 | 701.0 | ND | 297.1 |
| 446 | 634.5 | ^{a}ND | ND | ND | ND | 18.1 |

^{a}ND: Not detected;
^{b}AUC: Area under the curve

TABLE 2b
Fluorescence data for PpIX (5 μM) in EtOH
Solvent: EtOH

| λex | Emission maxima (nm) | | | | | |
|---|---|---|---|---|---|---|
| (nm) | Peak 1 | Peak 2 | Peak 3 | Peak 4 | Peak 5 | ^{b}AUC |
| 368 | 635.0 | 660.5 | 699.5 | ND | ND | 53.5 |
| 403 | 634.5 | 660.5 | 687.5 | 695.5 | 703.5 | 129.7 |
| 408 | 634.5 | 664.0 | 677.0 | 687.0 | 697.5 | 117.3 |
| 416 | 634.5 | 664.0 | 683.0 | 697.0 | 704.0 | 68.5 |
| 417 | 634.5 | 663.0 | 684.5 | 702.0 | ND | 63.9 |
| 446 | 634.5 | ND^{a} | ND | ND | ND | 2.2 |

^{a}ND: Not detected;
^{b}AUC: Area under the curve

TABLE 2c
Fluorescence data for PpIX (5 μM) in PBS
Solvent: PBS

| λex (nm) | Emission maxima (nm) | ^{b}AUC |
|---|---|---|
| 368 | 622.0 | 2.5 |
| 403 | 622.0 | 8.4 |
| 408 | 623.0 | 5.0 |
| 416 | 623.0 | 4.5 |
| 417 | 621.0 | 0.6 |
| 446 | ^{a}ND | ^{c}NA |

TABLE 2d
Fluorescence data for PpIX (5 μM) in FCS
Solvent: FCS

| λex | Emission maxima (nm) | | | | | |
|---|---|---|---|---|---|---|
| (nm) | Peak 1 | Peak 2 | Peak 3 | Peak 4 | Peak 5 | ^{b}AUC |
| 368 | 637.0 | 664.0 | 673.5 | 684.5 | 691.0 | 46.7 |
| 403 | 636.5 | 669.0 | 677.0 | 688.0 | 695.5 | 70.3 |
| 408 | 637.0 | 672.0 | 688.0 | ND | ND | 60.9 |
| 416 | 637.5 | 661.5 | 670.5 | 676.5 | ND | 44.6 |
| 417 | 637.0 | 676.5 | ND | ND | ND | 39.4 |
| 446 | 637.5 | ^{a}ND | ND | ND | ND | 4.7 |

^{a}ND: Not detected;
^{b}AUC: Area under the curve

TABLE 2e
Fluorescence data for PpIX in Liposomes
PpIX in Liposomes (1 mg/mL) (molar ratio of lipid:porphyrin was 75:1)

| λex | Emission maxima (nm) | | | | | | |
|---|---|---|---|---|---|---|---|
| (nm) | Peak 1 | Peak 2 | Peak 3 | Peak 4 | Peak 5 | Peak 6 | ^{c}AUC |
| 368 | 637.5 | 685.5 | 692.0 | 705.0 | ^{b}ND | ^{b}ND | 224.2 |
| 403 | 637.5 | 685.5 | 692.5 | 698.0 | 706.5 | ^{b}ND | 249.3 |
| 408 | 637.5 | 684.5 | 692.0 | 696.5 | 704.5 | ^{b}ND | 244.3 |
| 416 | 637.5 | 664.5 | 671.0 | 688.0 | 695.5 | 705.5 | 243.4 |
| 417 | 637.5 | 668.5 | 694.0 | 704.5 | 708.5 | ^{b}ND | 239.6 |
| 446 | 638.0 | 672.0 | 686.0 | 700.5 | 706.0 | ^{b}ND | 60.7 |

^{b}ND: Not detected;
^{c}AUC: Area under the curve

The amount and type of both endogenous porphyrin, and porphyrin synthesised from a porphyrin prodrug varies in different cell lines as can be seen in Tables 3 & 4.

TABLE 3
Baseline concentration of intracellular porphyrins.

| | Intracellular porphyrins (nM/million cells) | | |
|---|---|---|---|
| | Uroporphyrin | Coproporphyrin | Protoporphyrin |
| HaCaT | BLD | BLD | BLD |
| OE19 | BLD | BLD | BLD |
| HT1197 | BLD | 8.7 ± 0.1 | 0.5 ± 0.2 |
| SHSY5y | BLD | BLD | BLD |

HaCaT cells are an immortalised human skin keratinocytes cell line. OE19 are from a human oesophageal adenocarcinoma. HT1197 are from a human bladder carcinoma. SHSY5y are from a human neuroblastoma. BLD: below the limits of detection. Porphyrin isomers were detected by HPLC (cex: 405 nm; cem: 620 nm).

TABLE 4
Concentration of cellular porphyrins after treatment
with the porphyrin prodrug 5-aminolaevulinic acid.

| | Uroporphyrin | Coproporphyrin | Protoporphyrin |
|---|---|---|---|
| | Intracellular porphyrins (nM/million cells) | | |
| HaCaT | 0.6 ± 0.6 | 0.1 ± 0.2 | 3.5 ± 2.9 |
| OE19 | 1.7 ± 0.4 | 0.5 ± 0.4 | 13.4 ± 2.1 |
| HT1197 | 0.9 ± 0.2 | 4.4 ± 0.2 | 41.9 ± 10.8 |
| SHSY5y | Trace | 0.1 ± 0.1 | 4.9 ± 0.7 |
| | Extracellular porphyrins (nM) | | |
| HaCaT | 0.2 ± 0.1 | 0.5 ± 0.2 | 11.9 ± 3.2 |
| OE19 | 1.4 ± 0.9 | 7.2 ± 1.3 | 12.2 ± 5.5 |
| HT1197 | 1.3 ± 0.6 | 14.8 ± 4.8 | 120.1 ± 34.6 |
| SHSY5y | BLD | 0.3 ± 0.2 | 2.9 ± 0.3 |

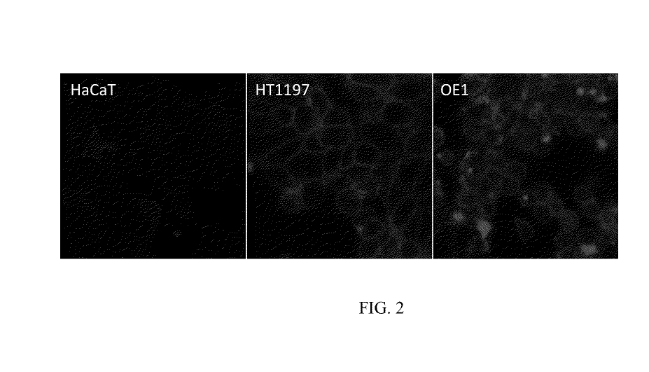

The intracellular environment of the porphyrins is also different, as illustrated in FIG. 2.

In the bladder cells the porphyrin may be more localized to the plasma membrane whereas in the other cell lines the signal is more diffuse and generally spread through the cytoplasm.

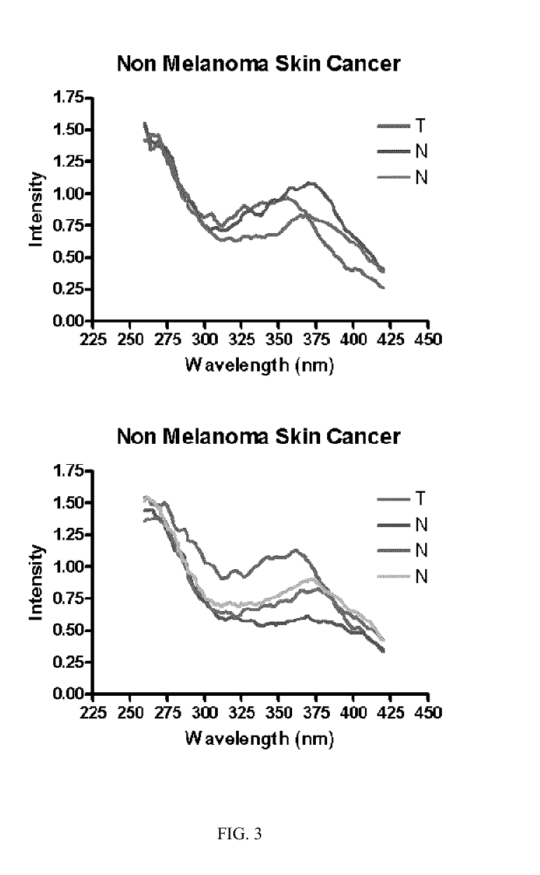

Other fluorophores contribute to tissue fluorescence. These endogenous fluorophores may not be uniformly distributed in the tissue, especially in stratified organs like skin and oesophagus. These fluorophores include structural components such as cyclic amino acids; and molecules involved in metabolism and physiology such as reduced pyridine nucleotides. Each species has a characteristic spectrum so theoretically these signals could be used to detect pathological changes. FIG. 3 shows the distinction between a nonmelanoma skin cancer and sounding normal tissue in two individuals. The spectrum obtained from the tumor is shifted compared to the normal tissue. By altering the excitation wavelength using a tunable device, the penetration of light into the tissue is changed, thus the collected signal can provide structural and metabolic information from different depths.

By using a combination of endogenous fluorescence and exogenous fluorescence produced by administration of a porphyrin prodrug it is possible to distinguish between normal tissue and diseased tissue; and also to distinguish between cancerous and non-cancerous, inflamed tissue and/or benign hyperproliferative disease.

### Use of Multiple Lasers

The use of a single tunable laser to tune an excitation wavelength has a number of limitations. A tunable laser in the 400 nm (blue/near UV) wavelength range is difficult and expensive to build. Also, with a single laser only the shift in absorption wavelength is detected, while information about emission wavelength or intermediate energy levels relevant for the fluorescence is not available. All wavelengths are affected by the local conditions and could thus provide useful information for cancer detection.

A combination of two or more lasers may be applied to affect the tissue fluorescence. The two beams may be co-located on a fiber or fiber bundle for endoscopic application, or they may be applied from different angles/distances.

A green laser and a near infrared laser are used to excite the protoporhyrin. Only when both the green wavelength and the infrared laser wavelengths are tuned correctly will there be an efficient excitation of the material and a red emission maximum. The red emission wavelength is also of interest. It may be detected by a spectrometer, however, a preferred approach is to utilize a tunable red laser to detect the emission wavelength either by detecting interference between fluorescence emission and the laser or by using the laser to cause stimulated emission from the excited material. Such stimulated emission will be generated only when the laser wavelength matches the fluorescence wavelength.

One or more of the lasers may be modulated. For instance if at least one of the excitation lasers is modulated, the emission is also modulated. The modulation in the emission can be detected directly. In a preferred implementation the modulation of stimulated emission is detected by mixing emitted light with the red laser used to generate the stimulated emission. Such a detection scheme is also capable of determining lifetime of the excited states of the protoporphyrin. By modulating the excitation lasers the material is also excited. Due to finite lifetime of the excited states this will decay.

The intensity of at least one laser may be modulated at a rate less than 10 GHz. The intensity of at least two lasers may be modulated at a rate less than 10 GHz, and the timing of the intensity maximum of the two lasers offset by at least 100 ps.

When a probe laser is used to generate stimulated emission, this emission will be detectable if the probe laser is turned on within the decay time of an excitation pulse the stimulated emission will be detected. If the delay between excitation and probe pulses is significantly larger than the decay time then the stimulated emission is not detected. Thus by tuning the delay between excitation and probe laser pulses different interference patterns can be created and the variation thereof can be detected. This can be done using fast detectors, but in a preferred embodiment this is done using a camera system or visually via a fiberscope. In this embodiment the delay between excitation and measurement pulse (which in itself will be short and fast in the ns range) is varied at a slow rate (in the ms to s range). As a result a varying interference pattern is created that stands out in areas with affected tissue. By choosing a slow rate in a range of 20 Hz or less a blinking pattern can result that is very visible.

Thus, the delay between the intensity peak of a first and a second laser may be modulated.

The modulation rate of the delay may be less than 1 kHz. Stimulated emission from the excited tissue is detected a laser tuned to the emission wavelength, and the stimulated emission intensity is monitored. Interference between the stimulated emission and the emission of the laser tuned to the stimulated emission is detected.

In another preferred embodiment a combination of a green and an infrared laser is used where the intensity of the infrared laser is modulated at a slow rate. The resulting emission variation in the red spectrum also results in a blinking pattern that enables easy detection of affected tissue. While the green excitation laser intensity may be constant and some of that intensity may be visible through a fiberscope this will result in a blinking variation of perceived color in the affected areas.

In another embodiment excitation of the material with both a blue laser in the 400 nm range and a green laser in the 500 nm range are used to detect emission at a red wavelength in the 600 nm range. Each wavelength can be tuned and modulated to establish the absorption spectrum and/or emission time constants at the wavelengths. A red laser can be tuned to establish the emission spectrum such that information about absorption and emission spectra in each wavelength range can be obtained and of the relevant fluorescence time constants can be determined.

Thus, at least two lasers can be used to detect tissue fluorescence, where the emission of at least one of the lasers is adjusted to detect emission at that wavelength or another wavelength. In one design, a UV/blue laser is used to excite the tissue with a wavelength near 400 nm. The intensity of the UV/blue laser is modulated and the intensity of emission at a longer wavelength is detected. Detection may be accomplished using a red laser with a wavelength near 600 nm, and detection of interference between emission and the red laser. A green laser with a wavelength around 500 nm can be used in combination with an infrared laser with a wavelength around 1500 nm.

Excitation of the tissue may be accomplished with an UV/blue laser in the 400 nm range. A green laser in the 500 nm range may used combined with a red laser in the 600 nm range to detect stimulated emission at the red wavelength.

Pathology in tissue may be identified using fluorescence in model biological systems, cell lines, biopsy and endoscopic resection samples. A fluorescent signal that permits differentiation between healthy and cancerous tissue (a ‘diagnostically adequate’ signal) may be produced from one or more dynamic blue prototype light sources. A dynamic blue prototype light source is a blue laser with at settable wavelength between 369 and 416 nm. All light sources—with excitation wavelengths ranging between 369-416 nm—successfully generate ALA-induced PpIX fluorescence signals. PpIX fluorescence is generated by employing multiple excitation wavelengths; however, irrespective of cell or tissue type, a shift in fluorescence emission wavelength of the main emission peak is not observed and remains anchored at approximately 636 nm. This highlights differences between protoporphyrin produced endogenously from endogenous ALA, preformed protoporphyrin added exogenously, and protoporphyrin produced exogenously from exogenous ALA. The endogeneous tests are inside the body, the exongeneous tests are outside the body. ALA may be used to produce protoporphyrin but is not required. In exogeneous tests with ALA, the main emission peak shifts as a function of the cell environment to an extent that cancer can be detected using this technique. Endogeneous tests do not necessarily exhibit this behavior. PpIX fluorescence intensity is time-dependent (i.e., 0-28 hours) after administering of ALA, regardless of OE19 cell growth conditions, for example, as either an in vitro monolayer or after transplantation to a chorioallantoic membrane (CAM) model.

A notable fluorescence emission peak occurs at approximately 620 nm for OE19 cancer cells in exogeneous conditions, which may indicate the presence of hydrophilic porphyrins, (coproporphyrin). The 620 nm peak intensity increases in a time-dependent manner after administering of ALA, concomitant with an increase at 636 nm, a spectroscopic effect supported by HPLC-FL analysis. In addition, the presence of the 620 nm is also linked to the appearance of a 680 nm peak. Different trends occur in the PpIX fluorescence ratio I₆₂₀/I₆₃₆; The use of alternative test set-ups with non-identical laser wavelengths and intensities may complicate the reproduction of results. The 620 nm peak, which changes in intensity with excitation wavelength, may be applied to aid the detection and differentiation between cancerous and normal cell types.

Dynamic blue prototype light sources are capable of detecting fluorescence differences between different types of cell lines, and between non-dysplastic and dysplastic Barrett's tissue. However, presently no significant inter-patient differences are found to distinguish between normal Barrett's epithelium from dysplastic tissue. Thus, the dynamic blue devices may be further optimized to increase clinical discriminatory value.

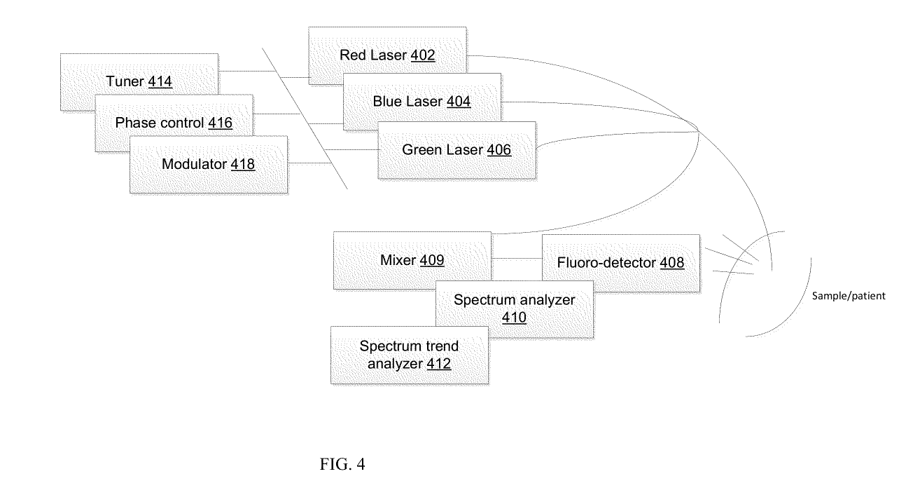

FIG. 4 illustrates an embodiment of a diagnostic system to carry out procedures described herein. One or more lasers, for example in the most versatile system red 402, green 406, and blue 404 spectrum lasers, are arranged to project light into a sample or patient. This may be done via an optical waveguide (shown linking laser outputs to the sample/patient), or via other light delivery mechanisms as described or known to practitioners in the art. One or more of the lasers may respond to controls to tune their wavelengths (414), to control the phase (timing) of intensity modulation between the lasers (416), or to modulation of output intensity or duration, or both (418). Fluorescence from the test site is detected (408) and (in some cases) mixed with incident excitation light (409). The output spectrum (optionally mixed with the input spectrum) may be analyzed (410) for intensity peaks, interference, and other indicia as described herein. The spectrum may be analyzed over time, to determine changes and trends that are indicia of pathologies (412), as described herein.

Logic may be incorporated into a control system that includes tuner 414, phase control 416, modulator 418, mixer 408, spectrum and trend analyzers 410, 412 to carry out the different diagnostic procedures and variants thereof which will now be apparent to those of skill in the art in light of this disclosure.

### Implementations and Alternatives

Those having skill in the art will appreciate that there are various logic implementations by which processes and/or systems described herein can be effected (e.g., hardware, software, and/or firmware), and that the preferred vehicle will vary with the context in which the processes are deployed. “Software” refers to logic that may be readily readapted to different purposes (e.g. read/write volatile or nonvolatile memory or media). “Firmware” refers to logic embodied as read-only memories and/or media. Hardware refers to logic embodied as analog and/or digital circuits. If an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations may involve optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood as notorious by those within the art that each function and/or operation within such block diagrams, flowcharts, or examples can be implemented, individually and/or collectively, by a wide range of hardware, software, firmware, or virtually any combination thereof. Several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory.

In a general sense, those skilled in the art will recognize that the various aspects described herein which can be implemented, individually and/or collectively, by a wide range of hardware, software, firmware, or any combination thereof can be viewed as being composed of various types of “circuitry.” Consequently, as used herein “circuitry” includes, but is not limited to, electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), circuitry forming a memory device (e.g., forms of random access memory), and/or circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into larger systems. That is, at least a portion of the devices and/or processes described herein can be integrated into a network processing system via a reasonable amount of experimentation.

The foregoing described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

What is claimed is:

1. A laser diagnostic system, comprising:
multiple lasers providing incident light on a sample, the light modulated and cooperatively phased to stimulate red emissions from the sample;
a detector coupled to a spectrum analyzer, to detect both the incident light and the red emissions from the sample, and to provide a signal indicating detection of a pathology in the sample.
